(19) United States
(12) Patent Application Publication
(10) Pub. No.: US 2006/0211624 A1
(43) Pub. Date: Sep. 21, 2006

(54) Methods and compositions for the treatment of parkinson's disease and other alpha-synucleinopathies

(75) Inventors: M. Maral Mouradian, Princeton, NJ (US); Eunsung Junn, East Brunswick, NJ (US)

Correspondence Address: EDWARDS ANGELL PALMER & DODGE LLP;(CLIENT REFERENCE NO. 47992), PO BOX 55874, BOSTON, MA, 02205, US

(73) Assignees: Goverment of the US, as Represented by; the Secretary, Department of Health and Human ology Transfer, Rockville, MD (US)

(21) Appl. No.: 10/544,326

(22) PCT Filed: Jan. 30, 2004
(86) PCT No.: PCT/US04/02724

§ 371 Date: Mar. 6, 2006

Related U.S. Application Data
(60) Provisional application No. 60/444,563, filed on Feb. 2, 2003.

(51) Int. Cl. A61K 38/08; A61K 38/06; A61K 31/4172; A61K 31/165; A61K 31/13

(52) U.S. Cl. 514/15; 514/16; 514/17; 514/18; 514/665; 514/617; 514/400

(57) ABSTRACT

The present invention provides novel methods for the treatment of Parkinson's disease and other α-synucleinopathies. The methods of the invention include treatment with inhibitors of transglutaminase, which can inhibit aggregation of α-synuclein. Also provided are screening assays for novel inhibitors of transglutaminase which may be used in the treatment of Parkinson's disease and other α-synucleinopathies.

### RELATED APPLICATIONS

This application claims the benefit of U.S. Provisional Application Ser. No. 60/444,563, filed Feb. 2, 2003, entitled “Methods and Compositions for the Treatment of Parkinson's Disease and Other α-Synucleinopathies”, the entire contents of which are hereby incorporated by this reference.

### BACKGROUND OF THE INVENTION

1. Field of the Invention

The invention provides, inter alia, new methods for treating a mammal suffering from a α-synucleinopathy such as Parkinson's disease by administration of a tTGase inhibitor compound or composition.

2. Background

Parkinson's disease (PD) is a neurodegenerative disorder characterized by the loss of dopaminergic neurons in the substantia nigra pars compacta and the presence of proteinaceous cytoplasmic inclusions known as Lewy bodies (I. Jenner, P. and Olanow. C. W. (1998) Ann. Neurol. 44:S72-S84; Pollimen, M. S. et al. (1993) J. Neuropathol. Exp. Neurol. 52:183-191). These inclusions are also present in dementia with Lewy bodies (DLB) (Gomez-Tortosa, E. et al. (2000) Acta Neuropathol. 99:352-357). Several lines of evidence point to a key role for α-synuclein in the pathogenesis of these disorders, which is a major constituent of Lewy bodies and whose mutations have been associated with rare autosomal dominant forms of PD (Spillantini, M. G. et al. (1998) Proc. Natl. Acad. Sci. USA 95:6469-6473; Polymeropoulos, M. H. et al. (1997) Science 276:2045-2047; Kruger. R. et al. (1998) Nat. Genet. 18:106-108; Mouradian, M. M. (2002) Neurology 58:179-185).

α-Synuclein is a relatively small protein of 140 amino acids consisting of three modular domains, including an N-terminal lipid-binding amphipathic α-helix, a central amyloid-binding domain encoding the non-AP component of Alzheimer plaques, and a C-terminal acidic tail (Riess, O. et al. (1998) Mol. Med. Today 4:438-444). α-Synuclein exists in either a natively unfolded conformation (Weinreb, P. H. et al. (1996) Biochemistry 35:13709-13715) or as an α-helix in the presence of phospholipid vesicles (Davidson. W. S. et al. (1998) J. Biol. Chem. 273:9443-9449), suggesting a dynamic regulation of its function depending on the local cellular environment. Because of its unfolded structure, α-synuclein is prone to self-aggregate and to cause the aggregation of other proteins, a property that may underlie its involvement in Lewy body formation and its contribution to the pathogenesis of PD (Conway, K. A. et al. (1998) Nat. Med. 4:1318-1320; Giasson. B. I. et al. (1999) J. Biol. Chem. 274:7619-7622; Paik, S. R. et al. (1998) FEBS Lett. 421:73-76). In vitro, α-synuclein is capable of self-aggregating into fibrils in a time-, temperature-, pH-, and concentration-dependent manner (Giasson. B. I. et al. (1999) J. Biol. Chem. 274:7619-7622; Hashimoto, M. et al. (1998) Brain Res. 799:301-306). Other factors such as mutations, C-terminal truncation, metal ions, and oxidative stress have also been shown to increase α-synuclein aggregation in vitro (El-Agnaf, O. M. et al. (1998) FEBS Lett. 440:67-70; Crowther, R. A. et al. (1998) FEBS Lett. 436:309-312; Hashimoto, M. et al. (1999) Neuro Report 10:717-721).

Additionally, factors thought to play a role in PD increase α-synuclein aggregation in several cellular models. These include pathogenic α-synuclein mutations, oxidative stress, proteasomal impairment, mitochondrial defects, and interaction with other proteins, such as parkin and synphilin-1 (Ostrerova-Golts, N. et al. (2000) J. Neurosci. 20:6048-6054; Paxinou, E. et al. (2001) J. Neurosci. 21:8053-8061; Rideout. H. J. et al. (2001) J. Neurochem. 78:899-908; Lee, H. J. et al. (2002) J Biol. Chem. 277:5411-5417; Junn, E. et al. (2002) J. Biol. Chem. 277:47870-47877; Engelender, S. et al. (1999) Nat. Genet. 22:110-114).

### SUMMARY OF THE INVENTION

We have now discovered that α-synuclein is a substrate for transglutaminase 2 (also referred to herein as ‘tTGase’) both in vitro and in cellular models.

We also have discovered that cystamine (also referred to herein as ‘CTM’), an inhibitor of tTGase, can inhibit tTGase-induced aggregation of α-synuclein, and that Lewy bodies in patients with Parkinson's disease (PD) and dementia with Lewy bodies (DLB) contain isodipeptide (i.e., tTGase-induced) cross-linked α-synuclein.

The invention provides methods of treating α-synucleinopathies (e.g., PD, DLB, and multiple system atrophy NSA)) and other neurodegenerative disorders comprising administering a therapeutically effective amount of a pharmaceutical composition comprising a tTGase inhibitor.

Suitable tTGase inhibitor compounds can be identified as disclosed herein. Particularly preferred tTGase inhibitors for use in therapies of the invention include cystamine, or a compound or composition that comprises cystamine (e.g., through covalent linkage, in an admixture, etc.). Other preferred tTGase inhibitor compounds for use in the therapies of the invention include monodansyl cadaverine, 1,3,4,5-tetramethyl-2-[(2-oxopropyl)thio]imidazolium chloride (L-682777), and peptides comprising one or more of the amino acid sequences RKLMEI (SEQ ID NO:3), GTLAKKLT (SEQ ID NO:4), SHLRKVFDK (SEQ ID NO:5), HDMNKVLDL (SEQ ID NO:6), MQMKKVLDS (SEQ ID NO:7), KVLD (SEQ ID NO:8), KVLDPVKG (SEQ ID NO:9), KVLDGQDP (SEQ ID NO:10), PVKG (SEQ ID NO:11), DPVKG (SEQ ID NO:12), and GQDP (SEQ ID NO:13).

Preferably, the methods of the invention prevent aggregation of α-synuclein and also prevent development and/or progression of symptoms of the α-synucleinopathy and/or and other neurodegenerative disorders.

In another embodiment, the invention provides methods of inhibiting α-synuclein aggregation in the cells of a subject suffering from or at risk for an α-synucleinopathy, comprising administering a therapeutically effective amount of a pharmaceutical composition comprising a tTGase inhibitor. Suitable cells are mammalian cells, particularly primate cells such as human cells. Suitable cells for treatment include neuronal cells and other mammalian cells.

In another embodiment, the invention provides methods (e.g., in vitro or in vivo methods) of identifying a compounds capable of treating an α-synucleinopathy comprising providing a composition comprising tTGase, contacting said composition with a test compound, and determining the ability of the test compound to inhibit tTGase activity, wherein a test compound capable of inhibiting tTGase activity is identified as a compound capable of treating an α-synucleinopathy. Preferably, tTGase activity is measured by determining the ability of tTGase to induce aggregation of α-synuclein.

Treatment methods of the invention include administration of one or more tTGase inhibitor compounds to a mammal suffering from or susceptible to an α-synucleinopathy or other neurodegenerative disorder. Preferably, the mammal is identified as suffering from or susceptible to an α-synucleinopathy or other neurodegenerative disorder and selected for treatment in accordance with the invention and then one or more tTGase inhibitor compounds are administered to the identified and selected mammal.

In a further aspect, the invention provides use of a tTGase inhibitor compound as disclosed herein for the treatment or prevention (including prophylactic treatment) of an α-synucleinopathy such as Parkinson's disease, and dementia with Lewy bodies or multiple system atrophy, or other neurodegenerative disorders such as Alzheimer's disease.

In a yet further aspect, the invention provides use of a of a tTGase inhibitor compound as disclosed herein for the preparation of a medicament for the treatment or prevention (including prophylactic treatment) of an α-synucleinopathy such as Parkinson's disease, and dementia with Lewy bodies or multiple system atrophy, or other neurodegenerative disorders such as Alzheimer's disease.

The invention also provides pharmaceutical compositions that comprise one or more tTGase inhibitor compounds together with a suitable carrier for the compounds. In such compositions, preferred tTGase inhibitor compounds include cystamine; or a compound or composition that comprises cystamine; monodansyl cadaverine; 1,3,4,5-tetramethyl-2-[(2-oxopropyl)thio]imidazolium chloride (L-682777); and peptides comprising one or more of the amino acid sequences RKLMEI (SEQ ID NO:3), GTLAKKLT (SEQ ID NO:4), SHLRKVFDK (SEQ ID NO:5), HDMNKVLDL (SEQ ID NO:6), MQMKKVLDS (SEQ ID NO:7), KVLD (SEQ ID NO:8), KVLDPVKG (SEQ ID NO:9), KVLDGQDP (SEQ ID NO:10), PVKG (SEQ ID NO:11), DPVKG (SEQ ID NO:12), and GQDP (SEQ ID NO:13).

Preferably, pharmaceutical compositions of the invention are packaged together with instructions (e.g. written instructions) for use of the composition to treat an α-synucleinopathy such as Parkinson's disease, and dementia with Lewy bodies or multiple system atrophy, or other neurodegenerative disorders such as Alzheimer's disease.

Other features and advantages of the invention will be apparent from the following detailed description and claims.

### BRIEF DESCRIPTION OF THE DRAWINGS

FIG. 1 depicts α-Synuclein the detection of aggregates by immunocytochemistry. HEK293T cells transiently transfected with α-synuclein in the absence or presence of tTGase were treated with CTM (100 μM, 200 μM) or A23187 (0.1 μg/ml) for 48 hours and stained for α-synuclein and tTGase. Aggregate-containing cells among transfected cells were counted in 10 randomly selected fields. Each microscopic field had 10-20 transfected cells. The data represent means±SEM. Significance levels determined by factorial ANOVA and the Bonferroni post hoc test are shown. *, P<0.002; * *, P<0.06; * * *, P<0.02.

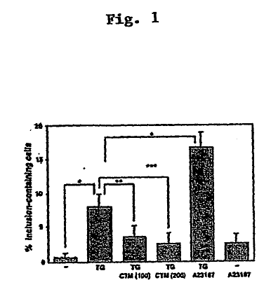

### DETAILED DESCRIPTION OF THE INVENTION

As discussed above, we have now discovered that α-synuclein is a substrate for transglutaminase 2 (also referred to herein as ‘tTGase’) both in vitro and in cellular models. Transglutaminases (TGases) are a family of proteins that catalyze a calcium-dependent transamidating reaction that results in cross-linking of proteins via ε(γ-glutamyl) lysine bonds (Greenberg, C. S. et al. (1991) FASEB J. 5:3071-3077). tTGase is unique in this family, in that it has GTPase and ATPase activities in addition to its transamidating activity (Achyuthan, K. E. and Greenberg, C. S. (1987) J. Biol. Chem. 262:1901-1906; Lai, T. S. et al. (1998) J Biol. Chem. 273:1776-1781). It is expressed in the mammalian nervous system and human brain, localizing predominantly in neurons (Kim. S. Y. et al. (1999) J. Biol. Chem. 274:30715-30721; Lesort, M. et al. (1999) J. Neurochem 73:2018-2027).

We also have discovered that formation of α-synuclein aggregates is significantly increased by tTGase activity. Moreover, tTGase-catalyzed cross-links colocalize with x-synuclein in Lewy bodies of Parkinson's disease (also referred to herein as ‘PD’) and dementia with Lewy bodies (also referred to herein as ‘DLB’). The present invention is still further based, at least in part, on the discovery that cystamine (also referred to herein as ‘CTM’), an inhibitor of tTGase, can inhibit tTGase-induced aggregation of α-synuclein, and that Lewy bodies in patients with PD and DLB contain isodipeptide (i.e., tTGase-induced) cross-linked α-synuclein.

As discussed above, methods are now provided for treating α-synucleinopathies, including PD and DLB, comprising administering tTGase inhibitors. The present invention further provides methods for identifying compounds capable of treating α-synucleinopathies, comprising identifying compounds which are tTGase inhibitors, and which, preferably, are inhibitors of tTGase-induced α-synuclein aggregation.

As use herein, the term ‘α-synucleinopathies’ (also sometimes referred to alternatively as ‘synucleinopathies’) includes diseases and/or disorders characterized by cellular aggregation of the protein α-synuclein. α-Synucleinopathies include, but are not limited to, PD, DLB, and multiple system atrophy (also referred to herein as ‘MSA’). In α-Synucleinopathies, aggregated α-synuclein is typically found as a major constituent of proteinaceous cytoplasmic inclusions known as Lewy bodies.

Suitable tTGase inhibitor compounds for use in the treatment methods and compositions of the invention can be assessed by straightforward protocols, such as the following. This following described protocol is referred to herein as ‘a standard in vitro tTGase inhibition assay’: Purified tTGase can be obtained from known sources, e.g., guinea pig liver, by methods known in the art (see, e.g., Leblanc, A. et al. (1999) Protein Expr. Purif. 17(1):89-95; also may be purchased from Sigma, St. Louis, Mo.). tTGase purified from guinea pig liver has a very broad substrate specificity in comparison with other members of the transglutaminase family and therefore is useful for substrate analogue kinetic studies. The assay is preformed in a buffer containing 50 mM Tris-HCl (pH 7.5), 2 mM leupeptin, and 1 mM x-synuclein. tTGase (10 nM) and DTT (0.1 mM) are added, the reaction is incubated at 37° C. for about 2 hours, and the reaction is stopped by the addition of 20 mM EDTA. The reaction products are then analyzed by standard SDS/PAGE and Western blot using an anti-α-synuclein antibody (e.g., rabbit polyclonal antibodies, available from Sigma or Chemicon (Temecula, Calif.); or monoclonal antibodies such as SYN-1 or LB509, available from Signal Transduction Laboratories (Lexington, Ky.) and Zymed (South San Francisco, Calif.), respectively). Cross-linking of α-synuclein by active tTGase results in the production of high-molecular weight (i.e., >60 kD) α-synuclein-containing aggregates. The assay can be preformed in the presence or absence of a candidate compound to determine whether the candidate compound can inhibit the production of the high-molecular weight aggregates. This defined standard in vitro tTGase inhibition assay is exemplified in Example 1 (including the materials and methods section) which follows.

Suitable tTGase inhibitor compounds for use in the treatment methods and compositions of the invention can be also be assessed by a protocol referred to herein as ‘a standard in vivo tTGase inhibition assay’, set forth as follows: Cells (e.g., neurons, COS-7 cells, or HEK 293K cells) are co-transfected with an α-synuclein and tTGase expression plasmids (1 μg each) using FuGene 6 reagent (Roche Molecular Biochemicals, Indianapolis, Ind.) for 6 hours and cultured in DMEM containing 10% FBS for 48 hours. Cells are then lysed in a buffer containing PBS with 1% Triton X-100 and a mixture of protease inhibitors (Roche Molecular Biochemicals). Cells are homogenized with 20 strokes in a Dounce homogenizer, centrifuged at 20,000×g at 4° C. for 30 minutes. The detergent-insoluble fraction is used in Western blot analysis using an anti-α-synuclein antibody as described above for the standard in vitro assay. The cells in the assay can be cultured in the presence or absence of a candidate compound to determine whether the candidate compound can inhibit the production of the high-molecular weight aggregates. This defined standard in vivo tTGase inhibition assay is exemplified in Examples 1 and 2 (including the materials and methods section) which follows.

The IC₅₀ (the concentration of the candidate compound required to provide 50% inhibition of tTGase catalyzed α-synuclein aggregation) can be determined using the standard assays described above. tTGase inhibitors generally suitable for the purposes of the invention will exhibit a detectable inhibition of the tTGase catalyzed α-synuclein aggregation in either of the above assays.

In one embodiment, the present invention provides methods of treating α-synucleinopathies (e.g., PD, DLB, and MSA) which comprise administering a therapeutically effective amount of a pharmaceutical composition comprising a tTGase inhibitor. In a preferred embodiment, the tTGase inhibitor is cystamine. Other preferred tTGase inhibitors include monodansyl cadaverine, 1,3,4,5-tetramethyl-2-[(2-oxopropyl)thio]imidazolium chloride (also referred to as L-682777), and peptide inhibitors, including peptides comprising one or more of the amino acid sequences RKLMEI (SEQ ID NO:3), GTLAKKLT (SEQ ID NO:4), SHLRKVFDK (SEQ ID NO:5), HDMNKVLDL (SEQ ID NO:6), MQMKKVLDS (SEQ ID NO:7), KVLD (SEQ ID NO:8), KVLDPVKG (SEQ ID NO:9), KVLDGQDP (SEQ ID NO:10), PVKG (SEQ ID NO:11), DPVKG (SEQ ID NO:12), and GQDP (SEQ ID NO:13) (see Sohn, J. et al. (2003) J. Clin. Invest. 111: 121-128, incorporated herein by reference). In one embodiment, the methods of the invention include administration of more than one tTGase inhibitor.

tTGase inhibitor compounds that comprise peptides preferably will have about 1000 or fewer amino acid residues, more preferably about 900, 800, 700, 600, 500, 400, 300, 200 or 100 or fewer amino acid residues. Relatively short peptides also will be suitable tTGase inhibitor compounds, particularly peptides having no more than about 90, 80, 70, 60, 50, 40, 30, 20 or even 15 or 10 amino acid residues, preferably including one or more of the following sequences:

| | |
|---|---|
| RKLMEI, | (SEQ ID NO:3) |
| GTLAKKLT, | (SEQ ID NO:4) |
| SHLRKVFDK, | (SEQ ID NO:5) |
| HDMNKVLDL, | (SEQ ID NO:6) |
| MQMKKVLDS, | (SEQ ID NO:7) |
| KVLD, | (SEQ ID NO:8) |
| KVLDPVKG, | (SEQ ID NO:9) |
| KVLDGQDP, | (SEQ ID NO:10) |
| PVKG, | (SEQ ID NO:11) |
| DPVKG, | (SEQ ID NO:12) |
| and | |
| GQDP. | (SEQ ID NO:13) |

To inhibit tTGase activity, and thereby inhibit α-synuclein aggregation, a tTGase inhibitor, e.g., a compound disclosed herein or identified by the screening assays of the invention, can be administered to a cell or a subject. Administration of a tTGase inhibitor to mammalian cells (including human cells, preferably neurons) can inhibit tTGase-mediated α-synuclein aggregation, thereby preventing accumulation of α-synuclein in Lewy bodies and inhibiting aggregate-related neurotoxicity and cell death. In such methods, the tTGase inhibitor can be administered to a mammal (including a human) by known procedures.

The preferred therapeutic methods of the invention (which include prophylactic treatment) in general comprise administration of a therapeutically effective amount of a tTGase inhibitor to an animal in need thereof, including a mammal, particularly a human. Such treatment will be suitably administered to subjects, particularly humans, suffering from, having, susceptible to, or at risk for an α-synucleinopathy (e.g., PD, DLB, or MSA), or other neurodegenerative disorder such as Alzheimer's disease, Down's Syndrome, Amyotrophic Lateral Sclerosis and Korsakoffs disease

The tTGase inhibitors of the invention may also be used in the treatment of other disorders in which α-synuclein and/or tTGase may be implicated, including, but not limited to, Alzheimer's disease as noted above and trinucleotide repeat expansion disorders (e.g., Huntington's disease, spinal and bulbar muscular atrophy, spinocerebellar ataxia type 1, dentatorubral-pallidoluysian atrophy, Machado-Joseph disease, spinocerebellar ataxia type 2, spinocerebellar ataxia type 6, and spinocerebellar ataxia type 7). Unless otherwise indicated, the term “α-synucleinopathy” as used herein is inclusive of such disorders in which α-synuclein and/or tTGase may be implicated, i.e. such as Alzheimer's disease and any of a variety of trinucleotide repeat expansion disorders.

For therapeutic applications, tTGase inhibitors of the invention may be suitably administered to a subject such as a mammal, particularly a human, alone or as part of a pharmaceutical composition, comprising the tTGase inhibitor together with one or more acceptable carriers thereof and optionally other therapeutic ingredients. The carrier(s) must be ‘acceptable’ in the sense of being compatible with the other ingredients of the formulation and not deleterious to the recipient thereof.

The pharmaceutical compositions of the invention include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well know in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Application of the subject therapeutics often will be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

Preferably, a pharmaceutical composition will be packaged together or otherwise in coordination with instructions for use of the pharmaceutical composition to treat a disease or disorder as disclosed herein. Typically, the instructions will be presented as written materials (e.g. package insert).

It will be appreciated that actual preferred amounts of a given tTGase inhibitor of the invention used in a given therapy will vary to the particular active compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests.

Screening Assays

The invention provides methods (also referred to herein as a ‘screening assay’) for identifying candidate or test compounds or agents (e.g., peptides, peptidomimetics, small molecules or other drugs) which inhibit tTGase activity. Such compounds are useful in the treatment of α-synucleinopathies, as discussed elsewhere herein. Preferably, a compound which is a tTGase inhibitor can inhibit the ability of tTGase to induce or mediate the aggregation of α-synuclein.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45). Examples of methods for the synthesis of molecular libraries can be found in the art, for example, in: DeWitt et al. (1993) Proc. Natl. Acad. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, a screening assay of the invention is performed in vivo, e.g., in a cell-based assay in which a cell which expresses tTGase is contacted with a test compound and the ability of the test compound to modulate tTGase activity is determined. Determining the ability of the test compound to modulate tTGase activity can be accomplished by monitoring, for example, whether tTGase is capable of inducing or mediating α-synuclein aggregation. In one embodiment, α-synuclein aggregation can be measured by lysing the cells and performing immunoprecipitation and Western blotting to determine whether the α-synuclein is in a monomeric or polymeric state. In another embodiment, the cells can be analyzed by immunocytochemistry to determine whether α-synuclein aggregates are present. In a preferred embodiment, the cell is a mammalian cell (e.g., a neuronal cell, a COS-7 cell, or a HEK 293K cell). Further exemplary methods can be found in the Examples section herein.

In another embodiment, a screening assay of the invention is preformed in vivo, e.g., in an animal that suffers from or is expected to develop an α-synucleinopathy, for example, a transgenic mouse which overexpresses α-synuclein. Test compounds can be administered to the animal to determine whether the compounds can inhibit aggregation of α-synuclein in the neurons of the animal and/or whether the compounds can inhibit development and/or progressions of α-synucleopathy symptoms.

In still another embodiment, a screening assay of the invention is performed in vitro. For example, a purified (i.e., cell-free) composition of tTGase can be contacted with a test compound, and the ability of the test compound to inhibit tTGase activity can be determined. tTGase activity can be measured, e.g., by determining the ability of the tTGase to mediate or induce the aggregation of x-synuclein.

Further in vivo and in vitro methods for measuring tTGase activity are described in the Examples section herein, and/or are known to those of skill in the art.

In another embodiment, tTGase inhibitors can be identified in a method wherein a cell is contacted with a candidate compound and the expression of tTGase mRNA or protein in the cell is determined. The level of expression of tTGase mRNA or protein in the presence of the candidate compound is compared to the level of expression of tTGase mRNA or protein in the absence of the candidate compound. The candidate compound can then be identified as a inhibitor of tTGase expression based on this comparison.

This invention is further illustrated by the following examples, which should not be construed as limiting. The contents of all references, patents, and published patent applications cited throughout this application, as well as the sequence listing and the figures, are incorporated herein by reference.

### EXAMPLES

Materials and Methods

The following materials and methods were used in Examples 1-6 below.

cDNA Cloning and Materials

α-Synuclein cDNA was cloned by PCR from human fetal brain cDNA library (Stratagene, La Jolla, Calif.) (Bennett, M. C. et al. (1999) J. Biol. Chem. 274:33855-33858) and subcloned into pcDNA3.1 (Invitrogen, Carlsbad, Calif.), and pTYB11 (New England Biolabs, Beverly, Mass.). tTGase cDNA was amplified by PCR from human liver cDNA library by using primers 5′-aagaattcAACAGGCGTGACGCCAGTTCTAAACTTGAAACAAAACAA-3′ (SEQ ID NO:1) and 5′-aagaattcGGAATTGTGTATTGCAAACATGGAGTGGAG-3′ (SEQ ID NO:2). Lowercase letters indicate additional nucleotides designed to facilitate cloning. The PCR product was inserted into pSG5 expression vector (Stratagene), and its sequence matched perfectly with the known cDNA sequence of human tTGase (Gentile, V. et al. (1991) J. Biol. Chem. 266:478-483). A catalytically inactive mutant of tTGase (C277S) was also used (Johnson, G. V. et al. (1997) Brain Res. 751:323-329; Tucholski, J. and Johnson, G. V. (2(1)2) J. Neurochem. 81:780-791). Mouse monoclonal tTGase antibody (CUB 7402) was purchased from DAKO (Carpinteria, Calif.). The rabbit polyclonal tTGase antibody was described in Kim. S. Y. et al. (1999) J. Biol. Chem. 274:30715-30721. Guinea pig liver tTGase, cystamine (CTM), and A23187 were purchased from Sigma (St. Louis, Mo.). Rabbit polyclonal α-synuclein antibodies were obtained from Sigma and Chemicon (Temecula, Calif.). Monoclonal anti-α-synuclein antibodies, SYN-1 and LB509, were from Transduction Laboratories (Lexington, Ky.) and Zymed (South San Francisco, Calif.), respectively. Monoclonal antibody recognizing ε(γ-glutamyl) lysine isodipeptide bonds (81D1C2) was purchased from Covalab (Lyon, France). Recombinant human α-synuclein protein was expressed in pTYB11 and purified by the IMPACT T7 system (New England Biolabs) according to the supplier's instructions.

Cell Culture and Transfection

COS-7 and human embryonic kidney (HEK) 293T cell lines were maintained in DMEM containing 10% FBS. Transfections were performed by using FuGene 6 reagent (Roche Molecular Biochemicals, Indianapolis, Ind.) for a 6 hour incubation period, and then treatments were initiated.

In Vitro Cross-Linking Reaction of α-Synuclein by tTGase

The reaction was performed at 37° C. for 2 hours in a buffer containing 50 mM Tris-HCl (pH 7.5), 2 mM leupeptin, and 1 mM purified α-synuclein. Depending on reaction conditions specified in the Examples below, purified guinea pig liver tTGase (10 nM), DTT (0.1 mM), CaCl₂ (5 mM), and/or EDTA (0.1 mM, 5 mM) were added. The reaction was stopped by the addition of 20 mM EDTA, and products were analyzed by SDS/PAGE followed by Western blot.

Immunoprecipitation and Western Blot

Cells were lysed in a buffer containing PBS with 1% Triton X-100 and a mixture of protease inhibitors (Roche Molecular Biochemicals). After homogenizing with 20 strokes by using a Dounce homogenizer, cells were centrifuged at 20,000×g at 4° C. for 30 min. The soluble and insoluble fractions were used in Western blot analysis using α-synuclein antibody (SYN-1) or tTGase antibody (CUB 7402). Triton X-100 insoluble pellets were dissolved in a buffer (PBS plus 1% Triton X-100/1% SDS) containing a mixture of protease inhibitors with sonication. After centrifugation, supernatant was diluted in 10 volumes of the same buffer lacking SDS and used for immunoprecipitation with SYN-1 antibody. Immunoprecipitates were analyzed by Western blots with antiisodipeptide antibody (81D1C2) or anti-α-synuclein antibody SYN-1 with the ECL detection system (NEN, Boston, Mass.). To study the interaction between α-synuclein and tTGase, cotransfected cells were lysed, and soluble fraction was subjected to immunoprecipitation with rabbit polyclonal tTGase antibody. Imnunoprecipitates or total cell lysates were analyzed by Western blots with SYN-1 antibody.

Immunocytochemistry

HEK 293T cells transiently transfected as described in the Examples below were cultured in the presence or absence of indicated chemicals in collagen-coated Biocoat slides (Becton Dickinson, Bedford, Mass.) for 48 hours. Cells were fixed in 4% formaldehyde in PBS for 20 min, washed with PBS three times, and permeabilized with 0.5% Triton X-100 in PBS for 10 min. After washing the cells again with PBS, they were blocked with 1% BSA in PBS for 20 min. Cells were incubated with rabbit polyclonal α-synuclein antibody (Sigma) and mouse monoclonal tTGase antibody (CUB 7402) diluted in 1% BSA at 4° C. for 2 hours. Cells were washed five times for 5 min each with PBS. Anti-rabbit IgG-rhodamine-conjugated and anti-mouse IgG-FITC-conjugated antibodies were diluted in 1% BSA and incubated at 4° C. for 1 hour. Cells were washed five times with PBS and analyzed under a fluorescence microscope (Axiophot, Zeiss, Thornwood, N.Y.). For quantification of inclusions, 10 microscopic fields were randomly selected, and the percentage of inclusion-positive cells was counted among transfected cells.

Immunohistochemistry on Human Brain Tissues

Postmortem brain tissues from patients with PD and DLB were fixed in formaldehyde for 2 weeks and embedded in paraffin. Six-micromolar sections from substantia nigra were immunostained individually with rabbit anti-α-synuclein polyclonal antibody (1:500, Chemicon) and mouse monoclonal isodipeptide antibody (81D1C2, 1:50) by using the avidin-biotin-peroxidase method as described (Lee, S. S. et al. (2002) Neurobiol. Aging, in press) with the Envision Plus kit (DAKO). Double immunohistochemistry with both antibodies was performed by using the Histostain-DS kit (Zymed) following the manufacturer's protocol. Colocalization of both signals in this kit is visualized under the light microscope as black color.

### Example 1

### α-Synuclein is a Substrate of tTGase In Vivo and In Vitro

To study the in vitro cross-linking of α-synuclein, purified recombinant human α-synuclein protein was incubated with guinea pig liver tTGase for 2 hours at 37° C., and the reaction products were analyzed by SDS/PAGE, followed by Western blotting with anti-α-synuclein antibody. In the presence of DTT, the reduced form of tTGase induced the formation of high molecular weight aggregates of α-synuclein. Generation of these aggregates was significantly enhanced by the addition of CaCl₂, a known activator of tTGase (Peterson, L. L. et al. (1983) J. Invest. Dermatol. 81:95-100). On the other hand, chelation with EDTA dose-dependently blocked aggregate formation. These results suggest that α-synuclein is a target for tTGase in vitro.

To examine tTGase-induced aggregation of α-synuclein in vivo, COS-7 cells were transfected with the α-synuclein expression plasmid (1 μg) in the absence or presence of a tTGase expression plasmid (0, 1, or 2 μg). The presence of tTGase dose-dependently induced the formation of α-synuclein high molecular weight aggregate bands running at apparent molecular masses >60 kDa in the detergent-insoluble fraction, detected by Western blot analysis with SYN-1 antibody. Similar results were obtained by using another α-synuclein antibody, LB509. The amount of these aggregates was much more abundant at 96 hours posttransfection than at 48 hours. In addition, expression of a catalytically inactive mutant (C277S) of tTGase failed to generate α-synuclein aggregates, indicating that their formation requires the catalytic activity of tTGase. Because ε(γ-glutamyl)lysine cross-links are the footprints of tTGase activity, the presence of isodipeptide bonds in these aggregates by immunoprecipitating α-synuclein from the detergent-insoluble fraction was determined. The complex immunoprecipitated from cells transfected with both α-synuclein and tTGase, but not from cells transfected with only α-synuclein, was detected by an antibody recognizing the isodipeptide bonds produced by tTGase activity. Additionally, there is a corresponding decrease in α-synuclein monomer as it is polymerized by tTGase. The latter data indicate that these aggregates are formed as a result of tTGase activity. The same findings were reproduced in HEK 293T cells transiently transfected with α-synuclein and tTGase. Collectively, these observations suggest that α-synuclein is a cellular substrate of tTGase.

### Example 2

### CTM Can Prevent the Formation of tTGase-Induced α-Synuclein Aggregates but Cannot Resolve Preexisting Complexes

Cystamine (CTM) is known to inhibit tTGase activity through a disulfide exchange reaction and serves as a competitor for tTGase by blocking access of the glutamine residue in substrate proteins to the active site of the enzyme (Birckbichler, P. J. et al. (1981) Proc. Natl. Acad. Sci. USA 78:5005-5008; Lorand, L. et al. (1979) Biochemistry 18:1756-1765). The ability of CTM to inhibit tTGase-induced α-synuclein aggregation in a cellular model was examined as described below. Incubation of α-synuclein- and tTGase-transfected COS-7 cells with 200 μM CTM for 48 hours dramatically inhibited the formation of these aggregates. To examine if CTM can clear preformed aggregates, cells were harvested at 48 hours and 96 hours posttransfection without CTM treatment, and also harvested at 96 hours after CTM treatment (200 μM) during the final 48 hours of incubation. Delayed treatment with CTM did not diminish the level of aggregates formed during the initial 48 hours. Rather, CTM inhibited further de novo aggregation occurring during the final 48 hours. This result suggests that CTM can prevent the formation of tTGase-induced α-synuclein aggregates but cannot resolve preexisting complexes.

### Example 3

### Calcium Ionophore Treatment Increases α-Synuclein Aggregation in a Cellular Model

The cross-linking activity of tTGase depends on calcium (Peterson, L. L. et al. (1983) J. Invest. Dermatol. 81:95-100), the effect of this cation on CL-synuclein aggregation was examined below. For this experiment, α-synuclein- and tTGase-transfected COS-7 cells were treated with the calcium ionophore A23187 (0.1 μg/ml). The calcium mobilizer resulted in a significant increase in the formation of α-synuclein aggregates. Notably, some high molecular weight bands of CL-synuclein were observed in A23187-treated cells transfected with only α-synuclein, likely caused by activation of endogenous tTGase despite its low expression in COS-7 cells or the tendency of CL-synuclein to form oligomers in the presence of calcium, as reported (Nielsen, M. S. et al. (2001) J. Biol. Chem. 276:22680-22684).

### Example 4

### α-Synuclein and tTGase Interact in Cells

To determine whether α-synuclein and tTGase interact, coimmunoprecipitation was performed with COS-7 cells transiently transfected with α-synuclein and tTGase. Immunoprecipitation of tTGase also pulled down α-synuclein, indicating intermolecular interaction. The inactive mutant (C277S) of tTGase also interacted with α-synuclein, suggesting that this interaction does not require the catalytic activity of the enzyme.

### Example 5

### CTM Can Prevent the Formation of tTGase-Induced α-Synuclein Aggregates in Cells as Determined by Immunocytochemistry

tTGase-induced α-synuclein aggregates could also be seen by immunocytochemistry in HEK293T cells transiently transfected with this enzyme and substrate. About 8% of cells expressing both proteins had microscopically visible aggregates, whereas only 0.7% of cells expressing only α-synuclein had inclusions. The very low level of α-synuclein aggregation into inclusions in the absence of tTGase overexpression likely represents the tendency of α-synuclein to aggregate (Conway, K. A. et al. (1998) Nat. Med. 4:1318-1320) because endogenous tTGase levels in these cells are quite low. These aggregates were localized in the cytosol, especially in the perinuclear region, and costained for tTGase. Consistent with the immunoblot analysis showing inhibition of aggregate formation, CTM (100 μM, 200 μM) resulted in decreased inclusion formation in cells coexpressing both proteins in a dose-dependent manner. A23187 (0.1 μg/ml), on the other hand, significantly increased aggregate formation, as seen above by immunoblot analysis.

### Example 6

### Lewy Bodies from PD and DLB Brains Contain Isodipeptide Cross-Linked α-Synuclein

To detect evidence for TGase activity in α-synucleinopathies, nigral sections from brains of patients affected with Parkinson's Disease (PD) and Dementia with Lewy Bodies (DLB) were subjected to immunohistochemical stains by using specific antibodies to isodipeptide (81D1C2) and α-synuclein. Lewy bodies from both disease conditions stained with 81D1C2 in the halo, similar to the well-known staining pattern of α-synuclein. Omission of the primary isodipeptide anti-body did not give an immunohistochemical signal. To confirm the colocalization of α-synuclein and isodipeptide cross-links, brain sections from PD and DLB were costained simultaneously with both primary antibodies, and adjacent sections were stained with hematoxylin and eosin. Colocalization of both signals was seen in the halo of Lewy bodies. These post-mortem studies indicate the presence of isodipeptide cross-linked α-synuclein in Lewy bodies from PD and DLB brains.

Those skilled in the art will recognize, or be able to ascertain using no more than routine experimentation, many equivalents to the specific embodiments of the invention described herein. Such equivalents are intended to be encompassed by the following claims.

1. A method of treating a mammal suffering from or susceptible to an x-synucleinopathy comprising administering to the mammal a therapeutically effective amount of a tTGase inhibitor.

2. The method of claim 1 wherein the tTGase inhibitor is cystamine or a compound or composition comprising cystamine.

3. The method of claim 1 wherein the tTGase inhibitor is selected from the group consisting of monodansyl cadaverine, 1,3,4,5-tetramethyl-2-[(2-oxopropyl)thio]imidazolium chloride (L-682777), a peptide comprising the amino acid sequence RKLMEI (SEQ ID NO:3), a peptide comprising the amino acid sequence GTLAKKLT (SEQ ID NO:4), a peptide comprising the amino acid sequence SHLRKVFDK (SEQ ID NO:5), a peptide comprising the amino acid sequence HDMNKVLDL (SEQ ID NO:6), a peptide comprising the amino acid sequence MQMKKVLDS (SEQ ID NO:7), a peptide comprising the amino acid sequence KVLD (SEQ ID NO:8), a peptide comprising the amino acid sequence KVLDPVKG (SEQ ID NO:9), a peptide comprising the amino acid sequence KVLDGQDP (SEQ ID NO:10), a peptide comprising the amino acid sequence PVKG (SEQ ID NO:11), a peptide comprising the amino acid sequence DPVKG (SEQ ID NO:12), and a peptide comprising the amino acid sequence GQDP (SEQ ID NO:13).

4. The method of claim 1 wherein the mammal is suffering or susceptible to Parkinson's disease.

5. The method of claim 1 wherein the mammal is suffering from dementia with Lewy bodies.

6. The method of claim 1 wherein the mammal is suffering from or susceptible to Alzheimer's disease or a trinucleotide repeat expansion disorder.

7. The method of claim 1 wherein the mammal is suffering from Huntington's disease, spinal or bulbar muscular atrophy, spinocerebellar ataxia type 1, dentatorubral-pallidoluysian atrophy, Machado-Joseph disease, spinocerebellar ataxia type 2, spinocerebellar ataxia type 6, or spinocerebellar ataxia type 7.

8. The method of claim wherein α-synuclein aggregation is inhibited in the mammal by administration of the tTGase inhibitor.

9. The method of claim 1 wherein development and/or progression of symptoms of the α-synucleinopathy is inhibited.

10. The method of claim 1 wherein the mammal is a primate.

11. The method of claim 1 wherein the mammal is a human.

12. (canceled)

13. A method of treating a mammal suffering from or susceptible to a neurodegenerative disease or disorder comprising administering to the mammal a therapeutically effective amount of a tTGase inhibitor.

14. The method of claim 13 wherein the tTGase inhibitor is cystamine or a compound or composition comprising cystamine.

15. The method of claim 13 wherein the tTGase inhibitor is selected from the group consisting of monodansyl cadaverine, 1,3,4,5-tetramethyl-2-[(2-oxopropyl)thio]imidazolium chloride (L-682777), a peptide comprising the amino acid sequence RKLMEI (SEQ ID NO:3), a peptide comprising the amino acid sequence GTLAKKLT (SEQ ID NO:4), a peptide comprising the amino acid sequence SHLRKVFDK (SEQ ID NO:5), a peptide comprising the amino acid sequence HDMNKVLDL (SEQ ID NO:6), a peptide comprising the amino acid sequence MQMKKVLDS (SEQ ID NO:7), a peptide comprising the amino acid sequence KVLD (SEQ ID NO:8), a peptide comprising the amino acid sequence KVLDPVKG (SEQ ID NO:9), a peptide comprising the amino acid sequence KVLDGQDP (SEQ ID NO:10), a peptide comprising the amino acid sequence PVKG (SEQ ID NO:11), a peptide comprising the amino acid sequence DPVKG (SEQ ID NO:12), and a peptide comprising the amino acid sequence GQDP (SEQ ID NO:13).

16. A method of inhibiting α-synuclein aggregation in the cells of a subject suffering from or at risk for an α-synucleinopathy, comprising administering a therapeutically effective amount of a tTGase inhibitor to the cells.

17. The method of claim 16 wherein the tTGase inhibitor comprises cystamine.

18. The method of claim 16 wherein the tTGase inhibitor is selected from the group consisting of monodansyl cadaverine, 1,3,4,5-tetramethyl-2-[(2-oxopropyl)thio]imidazolium chloride (L-682777), a peptide comprising the amino acid sequence RKLMEI (SEQ ID NO:3), a peptide comprising the amino acid sequence GTLAKKLT (SEQ ID NO:4), a peptide comprising the amino acid sequence SHLRKVFDK (SEQ ID NO:5), a peptide comprising the amino acid sequence HDMNKVLDL (SEQ ID NO:6), a peptide comprising the amino acid sequence MQMKKVLDS (SEQ ID NO:7), a peptide comprising the amino acid sequence KVLD (SEQ ID NO:8), a peptide comprising the amino acid sequence KVLDPVKG (SEQ ID NO:9), a peptide comprising the amino acid sequence KVLDGQDP (SEQ ID NO:10), a peptide comprising the amino acid sequence PVKG (SEQ ID NO:11), a peptide comprising the amino acid sequence DPVKG (SEQ ID NO:12), and a peptide comprising the amino acid sequence GQDP (SEQ ID NO:13).

19. The method of claim 16 wherein the α-synucleinopathy is Parkinson's disease.

20. The method of claim 16 wherein the α-synucleinopathy is dementia with Lewy bodies.

21. The method of claim 16 wherein development and/or progression of symptoms of the α-synucleinopathy is inhibited as a consequence of the administration of tTGase inhibitor.

22. The method of claim 16 wherein the cells are neuronal cells.

23. A method of identifying a compound capable of treating an α-synucleinopathy comprising:
a) providing a composition comprising tTGase;
b) contacting said composition with a test compound; and
c) determining the ability of the test compound to inhibit tTGase activity,
wherein a test compound capable of inhibiting tTGase activity is identified as a compound capable of treating an α-synucleinopathy.

24-36. (canceled)

37. A pharmaceutical kit comprising:
a pharmaceutical composition comprising a tTGase inhibitor; and
instructions for use of the pharmaceutical composition to treat an α-synucleinopathy.

38. The kit of claim 37 wherein the tTGase inhibitor is cystamine or a compound or composition comprising cystamine.

39. The kit of claim 37 wherein the tTGase inhibitor is selected from the group consisting of monodansyl cadaverine, 1,3,4,5-tetramethyl-2-[(2-oxopropyl)thio]imidazolium chloride (L-682777), a peptide comprising the amino acid sequence RKLMEI (SEQ ID NO:3), a peptide comprising the amino acid sequence GTLAKKLT (SEQ ID NO:4), a peptide comprising the amino acid sequence SHLRKVFDK (SEQ ID NO:5), a peptide comprising the amino acid sequence HDMNKVLDL (SEQ ID NO:6), a peptide comprising the amino acid sequence MQMKKVLDS (SEQ ID NO:7), a peptide comprising the amino acid sequence KVLD (SEQ ID NO:8), a peptide comprising the amino acid sequence KVLDPVKG (SEQ ID NO:9), a peptide comprising the amino acid sequence KVLDGQDP (SEQ ID NO:10), a peptide comprising the amino acid sequence PVKG (SEQ ID NO:11), a peptide comprising the amino acid sequence DPVKG (SEQ ID NO:12), and a peptide comprising the amino acid sequence GQDP (SEQ ID NO:13).

40. The kit of claim 37 wherein the instructions are for treatment of Parkinson's disease.


SEQUENCE LISTING (13 sequences)

SEQ ID NO: 1 - DNA, 47 bp, Artificial Sequence
  Description of Artificial Sequence Synthetic primer
aagaattcaacaggcgtgacgccagttctaaacttgaaacaaaacaa

SEQ ID NO: 2 - DNA, 38 bp, Artificial Sequence
  Description of Artificial Sequence Synthetic primer
aagaattcggaattgtgtattgcaaacatggagtggag

SEQ ID NO: 3 - protein, 6 aa, Artificial Sequence
  Description of Artificial Sequence Synthetic peptide
RKLMEI

SEQ ID NO: 4 - protein, 8 aa, Artificial Sequence
  Description of Artificial Sequence Synthetic peptide
GTLAKKLT

SEQ ID NO: 5 - protein, 9 aa, Artificial Sequence
  Description of Artificial Sequence Synthetic peptide
SHLRKVFDK

SEQ ID NO: 6 - protein, 9 aa, Artificial Sequence
  Description of Artificial Sequence Synthetic peptide
HDMNKVLDL

SEQ ID NO: 7 - protein, 9 aa, Artificial Sequence
  Description of Artificial Sequence Synthetic peptide
MQMKKVLDS

SEQ ID NO: 8 - protein, 4 aa, Artificial Sequence
  Description of Artificial Sequence Synthetic peptide
KVLD

SEQ ID NO: 9 - protein, 8 aa, Artificial Sequence
  Description of Artificial Sequence Synthetic peptide
KVLDPVKG

SEQ ID NO: 10 - protein, 8 aa, Artificial Sequence
  Description of Artificial Sequence Synthetic peptide
KVLDGQDP

SEQ ID NO: 11 - protein, 4 aa, Artificial Sequence
  Description of Artificial Sequence Synthetic peptide
PVKG

SEQ ID NO: 12 - protein, 5 aa, Artificial Sequence
  Description of Artificial Sequence Synthetic peptide
DPVKG

SEQ ID NO: 13 - protein, 4 aa, Artificial Sequence
  Description of Artificial Sequence Synthetic peptide
GQDP